(19) United States
(12) Patent Application Publication
(10) Pub. No.: US 2025/0128047 A1
(43) Pub. Date: Apr. 24, 2025

(54) INFLATION CARTRIDGE

(71) Applicant: ABIOMED, Inc., Danvers, MA (US)

(72) Inventor: Anthony Wong, Danvers, MA (US)

(73) Assignee: ABIOMED, Inc., Danvers, MA (US)

(21) Appl. No.: 18/923,902

(22) Filed: Oct. 23, 2024

Related U.S. Application Data
(60) Provisional application No. 63/545,227, filed on Oct. 23, 2023.
(60) Provisional application No. 63/558,465, filed on Feb. 27, 2024.

(51) Int. Cl. A61M 60/295; A61M 25/10; A61M 60/135; A61M 60/497

(52) U.S. Cl. CPC A61M 60/295; A61M 25/10182; A61M 25/10185; A61M 60/135; A61M 60/497

(57) ABSTRACT

An inflation cartridge may be provided. The cartridge may include a first and second syringe. The cartridge may include external housing defining an internal volume of space. The external housing may have at least one opening configured to allow access to an end of the first syringe and the second syringe. The housing may include a first port couplable to an expandable occlusion device. The housing may include a second port couplable to an external fluid source. The cartridge may include a plurality of valves operably coupled to the first syringe and the second syringe, the first port, and the second port.

### CROSS-REFERENCE TO RELATED APPLICATIONS

This application claims the benefit of U.S. Provisional Patent Applications Nos. 63/545,227, filed Oct. 23, 2023, and 63/558,465, filed Feb. 27, 2024. The disclosures of each of the foregoing applications are hereby incorporated by reference herein in their entirety.

### TECHNICAL FIELD

The present disclosure is drawn to blood flow-restricting devices, and specifically to cartridges for use with blood flow-restricting devices.

### BACKGROUND

If the pumping function of a patient's heart is insufficient despite other medical treatments, the circulatory system can be assisted in various ways. In some cases, a flow-restricting device (which may include, e.g., an inflatable balloon) may be installed into the patient's superior vena cava (SVC) to assist in regulating venous blood return to the heart. By adjusting the flow-restrictive device, the amount of blood flowing back to the heart can be controlled. However, preparing such devices for use can be challenging and time consuming.

### BRIEF SUMMARY

In various aspects, an inflation cartridge may be provided. The inflation cartridge may include a first syringe and a second syringe. The inflation cartridge may include an external housing. The external housing may define an internal volume of space. The external housing may have at least one opening configured to allow access to an end of the first syringe and the second syringe. The external housing may have a first port configured to be coupled to an expandable occlusion device. The external housing may have a second port configured to be coupled to an external fluid source. The inflation cartridge may include a plurality of valves operably coupled to the first syringe and the second syringe, the first port, and the second port.

In some embodiments, the plurality of valves may include a first valve coupled to the first syringe via a first channel. The plurality of valves may include a second valve coupled to the first valve via a second channel. The second valve may be coupled to the first port. In some embodiments, the plurality of valves may include a third valve. The third valve may be coupled to the second valve via a third channel. The third valve may be coupled to the second port. The third valve may be coupled to a fourth valve via a fourth channel. The fourth valve may be coupled to the second syringe. The first valve and the fourth valve may be coupled to a fluid sensor via one or more additional channels.

In some embodiments, the inflation cartridge may include one or more processors. The one or more processors may be configured to, collectively, control the inflation cartridge.

In some embodiments, the inflation cartridge may include one or more interfaces. The one or more interfaces may include at least one interface configured to allow the inflation cartridge to be operably coupled to a power source. The one or more interfaces may include at least one interface configured to allow the inflation cartridge to be operably coupled to one or more external processors. The one or more interfaces may include at least one interface configured to allow the inflation cartridge to be operably coupled to one or more optical fibers.

In various aspects, a system may be provided. The system may include an inflation cartridge as disclosed herein. The system may include a controller. The controller may be configured to removably receive the inflation cartridge.

In some embodiments, the external housing of the inflation cartridge may be configured to be received by the controller in only one orientation (e.g., the cartridge may be prevented from being received by the controller in an incorrect orientation). In some embodiments, the controller may include a first actuator. The first actuator may be configured to control the first syringe. The controller may include a second actuator. The second controller may be configured to control the second syringe.

In some embodiments, the system may further include an expandable occlusion device. The expandable occlusion device may be operably coupled to the inflation cartridge.

In some embodiments, the controller may include one or more processors. The one or more processors may be configured to, collectively, control the inflation cartridge. The controller may include a display. The display may be operably coupled to the one or more processor(s). The one or more processors may be configured to cause the inflation cartridge to receive fluid from a fluid source. In some embodiments, the fluid may be a liquid, such as a saline fluid. The fluid may include a contrast agent.

In some embodiments, the fluid received from the fluid source may flow into the first syringe through one or more valves of the plurality of valves. In some embodiments, the fluid may be flowed from the first syringe to the second syringe through at least one of the plurality of valves. In some embodiments, the fluid may be flowed from the first syringe to the second syringe through each of the plurality of valves.

In some embodiments, the one or more processors may be configured to cause the first syringe to draw air from an expandable occlusion device operably coupled to the inflation cartridge. In some embodiments the one or more processors are configured to cause the first syringe to purge air from the first syringe. In some embodiments, the one or more processors are configured to cause the fluid to flow from the second syringe to the first syringe. In some embodiments, the one or more processors are configured to cause the fluid to flow from the first syringe to the expandable occlusion device. In some embodiments, the one or more processors are configured to cause the fluid to flow from the second syringe to the expandable occlusion device.

In some embodiments, the inflation cartridge may be controlled by one or more processors, either processor(s) on the cartridge, processor(s) on the controller, or both. The processor(s) may control the flow of various fluids through the cartridge. The processor(s) may be configured to cause fluid received from the fluid source to flow into the first syringe through one or more valves. The processor(s) may be configured to cause the fluid to flow from the first syringe to the second syringe through at least one of the plurality of valves. In some embodiments, the fluid may flow from the first syringe to the second syringe through each of the plurality of valves.

In various aspects, a method for using an expandable occlusion device may be provided. The method may include receiving fluid in a first syringe from a fluid source, through one or more valves of a plurality of valves. The method may include flowing fluid from the first syringe to a second syringe through at least one of the plurality of valves. The method may include drawing air, with the first syringe, from an expandable occlusion device. The method may include flowing fluid to the expandable occlusion device.

In some embodiments, the fluid may be flowed from the first syringe to the second syringe through each of the plurality of valves.

In some embodiments, the first syringe, second syringe, and plurality of valves are within an external housing of an inflation cartridge.

In some embodiments, the method may include removably attaching the inflation cartridge to a controller. In some embodiments, the method may include removing the inflation cartridge from the controller after a predetermined period of time.

In some embodiments, the method may further include purging air from the first syringe. The method may include flowing the fluid from the second syringe to the first syringe.

In some embodiments, flowing the fluid to the expandable occlusion device may include causing the fluid to flow from the first syringe to the expandable occlusion device. In some embodiments, flowing the fluid to the expandable occlusion device may include causing the fluid to flow from the second syringe to the expandable occlusion device.

In some embodiments, the method may further include intermittently providing a saline flush to the expandable occlusion device.

In some embodiments of the method, the inflation cartridge may be disposed outside a sterile field. The expandable occlusion device may be disposed within a sterile field.

In some embodiments, the method may further include determining or confirming whether the expandable occlusion device was inserted into a blood vessel after drawing air and prior to flowing the fluid to the expandable occlusion device. In some embodiments, the method may further include flowing fluid from the expandable occlusion device to either the first or second syringe. In some embodiments, the method may include repeatedly flowing fluid into and out of the expandable occlusion device at predetermined times. In some embodiments, the method may include removing the expandable occlusion device from the blood vessel.

In various aspects, a kit may be provided. The kit may include an inflation cartridge as disclosed herein. The kit may include an expandable occlusion device. The kit may optionally include one or more fill lines for coupling the inflation cartridge to the expandable occlusion device.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration showing a device with a plurality of expandable occlusion elements within a patient.

FIG. 2 is an illustration of a top view of an inflation cartridge.

FIG. 3 is an illustration of a perspective view of an inflation cartridge with external housing.

FIG. 4 is an illustration of an inflation cartridge (left) and an exploded view thereof (right) according to some embodiments.

FIG. 5 is an illustration of an inflation cartridge removably attached to a controller.

FIG. 6 is an illustration of a portion of a controller of a medical device according to some embodiments.

FIG. 7 is an embodiment of an inflation cartridge slidably attached to a controller.

FIG. 8 is an embodiment of a controller configured to receive an inflation cartridge.

FIG. 9 is an embodiment of a controller configured to receive an inflation cartridge.

FIG. 10 is an embodiment of a controller with a pivotably mounted cover configured to receive an inflation cartridge.

FIG. 11 is an embodiment of an integrated workstation.

FIG. 12 is a flowchart of a method.

FIG. 13 is an embodiment of a kit.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

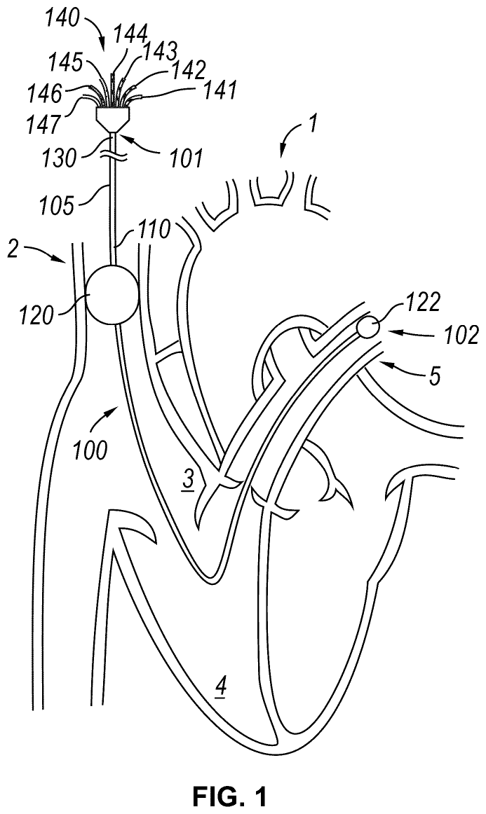

As seen in FIG. 1, in some cardiovascular treatments, a flow-restrictive device 100 may be disposed at least partially within a patient. Here, the device is shown as being inserted into a patient's heart 1, extending through the superior vena cava 2, right atrium 3, right ventricle 4, and into the pulmonary artery 5. The device is shown as including a catheter 105. The catheter may have a tubular body 110 with a first end 101 and a second end 102. The device may be one or more occlusive elements (which may be, e.g., balloons or other similar known occlusion elements). The device is shown as having two expandable occlusion elements coupled to the catheter—a first expandable occlusion element 120 (shown here disposed in the superior vena cava, at an intermediate point on the catheter between a first end 101 and second end 102 of the catheter), and a second expandable occlusion element 122 (which here disposed in the pulmonary artery, at a distal end of the catheter). There may be a portion 130 of the catheter that extends outside the patient's body. As shown, to control the device, there may need to be a number of connections 140 to the catheter. Here, we see seven connections, which may include, e.g., a guidewire 141, tubes 142, 143 for connecting a fluid source to the expandable occlusion element(s), wires or tubes 144, 145 for connecting to sensors in or on the device, tubes 146 for drawing fluid from the blood vessel, and a tube 147 for withdrawing air/fluid from one or both of the expandable occlusion elements (which may be operably coupled to a vacuum pump).

Given the complexity of the device, as seen in FIG. 1, and number of connections that may be required, conventional techniques for utilizing flow-restricting devices often include several dozen steps and may take almost an hour to complete before the patient can receive a needed treatment. Further, conventional techniques involve routinely passing components or devices into and out of the sterile field. The passage of components or devices into and out of the sterile field increases the risk of contamination and thus the risk of injury to the patient.

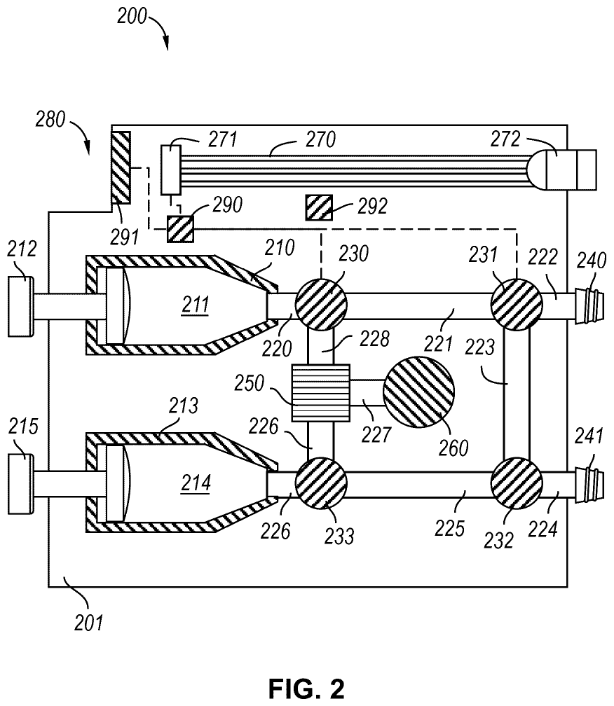

To improve on conventional techniques, in various aspects, an inflation cartridge may be provided. Referring to FIG. 2, the inflation cartridge 200 may include a supporting substrate 201. This may be any appropriate rigid material and may be a polymer.

The inflation cartridge may include a first syringe 210. The inflation cartridge may include a second syringe 213. The first and second syringes 210, 213 may each define an internal cavity 211, 214 for holding a fluid to be dispensed. The first and second syringes 210, 213 may each include a plunger 212, 215. As is understood in the art, the plunger 212, 215 may be depressed to cause fluid to exit the internal cavity 211, 214 and raised to draw fluid into the internal cavity 211, 214. As will be appreciated, any suitable number of syringes may be used. For example, the inflation cartridge may include a third syringe.

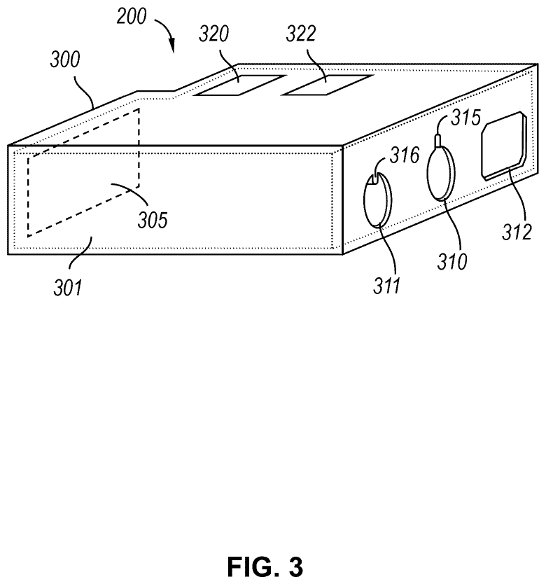

Referring to FIG. 3, the inflation cartridge may include an external housing 300. The external housing 300 may define an internal volume of space 301, in which at least some of the components of the inflation cartridge may be secured. The external housing may have at least one opening 305. The at least one opening 305 may be configured to allow access to an end of the first syringe 210 and the second syringe 213. The external housing 300 may have a first port 310. The first port 310 may be configured to be coupled to an expandable occlusion device. The external housing 300 may have a second port 311. The second port 311 may be configured to be coupled to an external fluid source. The external housing 300 may have a third port 312 configured to be coupled to one or more electrical or optical cables. Each port may have a different shape, or may have one or more features 315, 316 (e.g., protrusions, depressions, etc.) such that only a particular connector can be connected to the port. That is, the port configuration can be used to prevent a person from attaching incorrect tubing to a particular port. For example, the first port (which is meant to be coupled to the expandable occlusion device) may have a configuration which prevents a person from attaching tubing meant for the external fluid source.

As will be appreciated, although the inflation cartridge 200 shown in FIG. 3 has substantially quadrilateral cross-sectional shape, the inflation cartridge may have any suitable cross-sectional geometry. For example, the inflation cartridge may have a pentagonal, hexagonal, or heptagonal shape. Furthermore, the inflation cartridge may have any suitable geometry on the edges and/or corners. For example, the inflation cartridge may have rounded edges and/or corners.

The external housing 300 may include one or more visual indicators 320 (e.g., lights, display screens, etc.) to allow information about the cartridge to be passed to a user. For example, in some embodiments, the visual indicator may show one color when the cartridge is first attached to a controller. In some embodiments, the visual indicator may show another color when the cartridge is attached improperly to the controller. In some embodiments, the visual indicator may show a different color when the cartridge is first activated or used (e.g., when fluid, etc., first enters into the device). In some embodiments, the visual indicator may show a different color after the device has been used for a predetermined period of time. In some embodiments, the visual indicator may display a period of time remaining before the cartridge should be replaced. In some embodiments, the indicator may display a number of cycles the device has been used for. In some embodiments, the indicator may display a status code or description (e.g., “code 12”, “in use”, etc.).

In one embodiment, the visual indicator may display an instruction to a user. One such instruction may direct a user to load the inflation cartridge. In another embodiment, the visual indicator may direct a user to prepare a catheter. In yet another embodiment, the visual indicator may direct a user to refill a syringe. As will be appreciated, the visual indicator may display any number of useful instructions to a user (e.g., doctor, nurse, or technician).

The external housing 300 may include one or more buttons 322, knobs, etc. In some embodiments, the one or more buttons may be used to cause the visual indicator to display a time remaining before the cartridge should be replaced. In another embodiment, the buttons may be used to cause the depression of a syringe. In another embodiment, the buttons may be used to check a status of the cartridge. In some embodiments, the buttons or knobs are configurable to adjust a pressure in the valves or tubes. In another embodiment, the buttons or knobs are configurable to adjust a flow rate of the fluid to the expandable occlusion from the syringes. In some embodiments, the buttons or knobs are configured to acknowledge a visual indicator.

Referring to FIG. 2, the inflation cartridge may include a plurality of valves (230, 231, 232, 233) operably coupled to the first syringe 210 and the second syringe 213, the first port (here, a first port connector 240 is shown), and the second port (here, a second port connector 241 is shown).

The plurality of valves may include a first valve 230 coupled to the first syringe 210 via a first channel 220. The first valve may be coupled to a fluid sensor 250 via channel 228. As will be appreciated, any suitable fluid sensor 250 may be used. For example, the fluid sensor 250 may be an optical sensor. The fluid sensor can be configured to measure characteristics or parameters of fluid passing through the valves and channels. Examples of fluid characteristics or parameters that can be used for controlling the inflation cartridge include fluid pressure or flow volume.

The plurality of valves may include a second valve 231 coupled to the first valve 230 via a second channel 221. The second valve 231 may be coupled to the first port (e.g., via channel 222 connected to first port connector 240). The first port connector is shown as having a threaded exterior, but as will be understood, any appropriate means for connecting tubing may be utilized, including use of clamps, fittings, etc.

The plurality of valves may include a third valve 232 coupled to the second valve 231 via a third channel 223. The third valve 232 may be coupled to the second port (e.g., via channel 224 connected to second port connector 241).

The plurality of valves may include a fourth valve 233 coupled to the third valve 232 via a fourth channel 225. The fourth valve 233 may be coupled to the second syringe 213, e.g., via channel 226. The fourth valve 233 may be coupled to fluid sensor 250 via channel 226. As will be appreciated, any suitable fluid sensor may be used. For example, the fluid sensor may be an optical sensor. The fluid sensor can be configured to measure characteristics or parameters of fluid passing through the channels. Examples of fluid characteristics or parameters that can be used for controlling the inflation cartridge include fluid pressure or flow volume.

The fluid sensor 250 may be coupled to a valve or drain port 260 via channel 227.

The inflation cartridge may include one or more batteries 292. The batteries may be included to provide power to the inflation cartridge.

The inflation cartridge may include one or more processors 290 configured to, collectively, control the inflation cartridge. The inflation cartridge may include one or more interfaces 291, 272. The interface(s) may include at least one interface configured to allow the inflation cartridge to be operably coupled to a power source. The interface(s) may include at least one interface configured to allow the inflation cartridge to be operably coupled to one or more external processors.

The interface(s) may include at least one interface 272 configured to allow the inflation cartridge to be operably coupled to one or more optical fibers. The inflation cartridge may include one or more optical fibers 270 coupled to the interface 272. The inflation cartridge may include one or more receivers 271 configured to receive information from optical fiber(s) and output an electrical output signal. The receivers may be operably coupled to one or more processors 290. As will be understood, in some embodiments, rather than receiving and converting optical information in the cartridge, one or more optical fibers may simply “pass through” through the cartridge, instead of terminating at receiver 271, one or more optical fibers 270 may pass through to another optical interface (e.g., interface 291) where another optical fiber would be coupled to the cartridge.

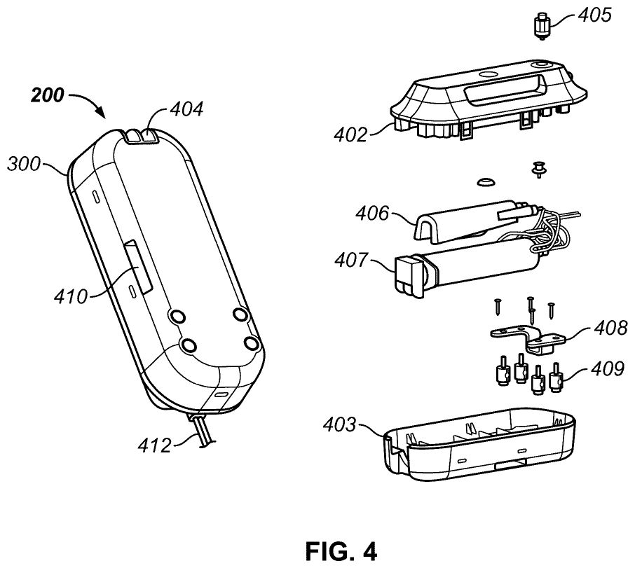

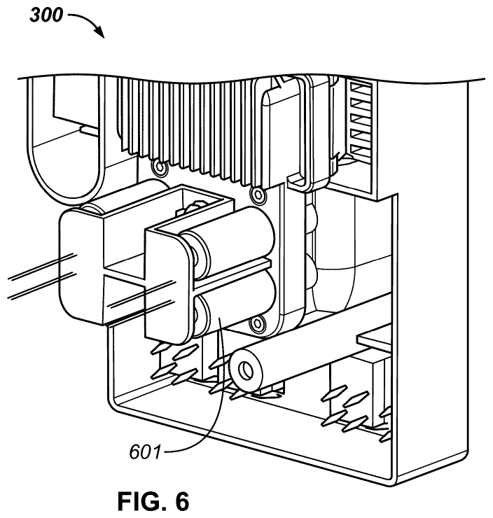

An example of an inflation cartridge 200 is shown in FIG. 4. Referring to FIG. 4, an inflation cartridge and an exploded view of the inflation cartridge is shown. The inflation cartridge 200 may be defined by an external housing 300 and an inner volume. The external housing 300 may further be defined by a front housing 402 and a back housing 403. The front housing 402 may include a syringe pump interface 404. Within the inner volume, the inflation cartridge 200 may include a luer-activated valve 405, reservoir bag 406, syringe 407, valve retainer 408, and guillotine valve buttons 409. The external housing may be opened via a latch mechanism 410. As with the other embodiments described herein, the cartridge may include a syringe 407 having one or more fluid lines and one or more valves. As shown in this view, the one or more valves may include guillotine valve buttons 409 that are configured to pinch (and or release) the one or more fluid lines to allow fluid to flow to inflate and/or deflate the balloon. In some embodiments, the valve buttons 401 may be disposed in a valve retainer 408 of the cartridge. In some embodiments, the inflation cartridge may include a plurality of wires 412 extending from the back portion of the external housing. As shown in FIG. 6, in some embodiments, the external housing 300, into which the cartridge is inserted, may include one or more actuators 601 configured to engage with the valve buttons to pinch (and/or release) the fluid lines. In some embodiments, the one or more actuators 601 may include solenoids configured to push on the valve buttons inside the cartridge to turn on and off the various valves (e.g., tube pathways). For example, in some embodiments, the solenoids may be configured to push the guillotine valve buttons 409 to achieve one or more cartridge states.

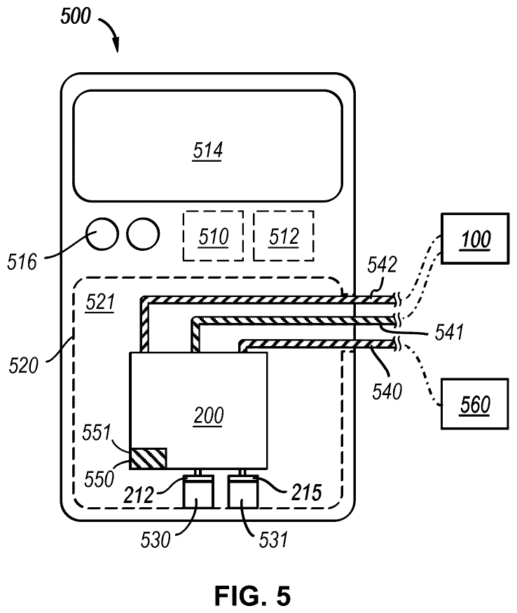

In some embodiments, as shown in FIGS. 5 and 6, the inflation cartridge may have 4 guillotine valve buttons 409 and the cartridge may have 4 corresponding actuators. As will be appreciated, any suitable number of valve buttons and actuators may be used. As will be further appreciated, the inflation cartridge may include a console solenoid interface (see, e.g., FIG. 5). In some embodiments, as described herein, the actuators may actuate one or more of the valve buttons 409 at the same time, such as to achieve the one or more inflation cartridge states. For example, two actuators may actuate two valve buttons (e.g., to pinch the fluid lines), while the two other actuators are disengaged. In another example, one actuator may actuate one valve button (e.g., to pinch one fluid line), while the other three actuators are disengaged.

In various aspects, a system may be provided. Referring to FIG. 5, the system may include an inflation cartridge 200 as disclosed herein. The system may include a controller 500 configured to removably receive the inflation cartridge. The housing of the inflation cartridge may be configured to be received by the controller 500 in only one orientation. Referring briefly to FIGS. 2 and 5, the cartridge may include one or more features 280, 550 (such as a notch, indentation, hole, etc.) that are configured to align with one or more features on the controller 500. In FIG. 5, feature 280 is a cutout corner of the cartridge that aligns with feature 550 on the controller, such that the cartridge can only be inserted correctly in one orientation, and the cartridge is otherwise prevented from being received by the controller 500 in an incorrect orientation. In some embodiments, feature 550 may also include an interface 551, which may be operably coupled to an interface on the cartridge (such as interface 291 shown in FIG. 2). As will be appreciated, although the feature 280 is shown adjacent to the indicator lights in FIG. 3, the feature 280 may be located in any suitable position on the inflation cartridge.

The controller may include a first actuator 530 configured to control the first syringe. In FIG. 5, the actuator is shown as being removably coupled to plunger 212 of the first syringe. The controller may include a second actuator 531 configured to control the second syringe. In FIG. 5, the actuator shown as being removably coupled to plunger 215 of the second syringe. Actuation of the first or second actuator may cause the plunger of the first or second syringe to be depressed.

The controller may include one or more processors 510 configured to, collectively, control the inflation cartridge. The controller 500 may include one or more non-transitory computer readable storage devices 512 containing instructions that, when executed by the one or more processors, control the performance of the controller 500. The controller 500 may include a display 514 operably coupled to the controller's processor(s). The controller 500 may include one or more buttons 516 operably coupled to the controller's processor(s). The buttons may be configured to adjust a parameter. The parameter may be a flow rate or a pressure of the occlusion device. The button may also be configured to act as an emergency evacuation switch. The emergency evacuation switch may initiate a protocol to remove the expandable occlusion device from a patient.

The controller's one or more processors may be configured to cause the inflation cartridge to receive fluid from a fluid source. The fluid may be a liquid, such as a saline fluid. The fluid may include a contrast agent.

The inflation cartridge may be controlled by one or more processors, either processor(s) 290 on the cartridge, processor(s) 510 on the controller, or both. The processor(s) may be configured to perform some or all of a method as disclosed herein. The processor(s) may control the flow of various fluids through the cartridge by controlling actuators of the controller 500 and/or one or more valves on the cartridge. In some embodiments, the flow through the cartridge is controlled only by processor(s) on the cartridge. In some embodiments, the flow through the cartridge is controlled only by processor(s) on the controller 500. In some embodiments, the flow through the cartridge is controlled by processor(s) on both the cartridge and the controller 500. In some embodiments, the controller may cause an actuator to press or depress a plunger of the first or second syringe.

The system may include an expandable occlusion device operably coupled to the inflation cartridge 200. In FIG. 5, this is shown as having one or more optical fibers 542 connecting the cartridge (e.g., via interface 272 in FIG. 2 and/or via third port 312 in FIG. 3) and the occlusion device. In some embodiments, the one or more optical fibers 542 may be the same as the one or more wires or tubes 144, 145 for connecting to sensors in or on the device as seen in FIG. 2.

The connection between the occlusion device and the inflation cartridge may also include tube 541, which may be coupled to a port or connector on the cartridge (e.g., first port 310 in FIG. 3 and/or first port connector 240 in FIG. 2).

The system may also include a fluid source 560. The fluid source may include a liquid, such a saline fluid. The fluid source may include a contrast agent. The fluid source may be coupled to the inflation cartridge via tube 540, which may be coupled to a port of connector on the cartridge (e.g., second port 311 in FIG. 3 and/or second port connector 241 in FIG. 2). As will be appreciated, any fluid suitable for filling either one or both of the first expandable occlusion element and the second expandable occlusion element may be used.

The processor(s) may be configured to cause fluid received from the fluid source to flow into the first syringe through one or more valves. The processor(s) may be configured to cause the fluid to flow from the first syringe to the second syringe through at least one of the plurality of valves. In some embodiments, the fluid may flow from the first syringe to the second syringe through each of the cartridge's valves. The processor(s) may be configured to cause the first syringe to draw air from an expandable occlusion device operably coupled to the inflation cartridge. The processor(s) may be configured to cause the first syringe to purge air from the first syringe. The processor(s) may be configured to cause the fluid to flow from the second syringe to the first syringe. The processor(s) may be configured to cause the fluid to flow from the first syringe to the expandable occlusion device. The processor(s) may be configured to cause the fluid to flow from the second syringe to the expandable occlusion device.

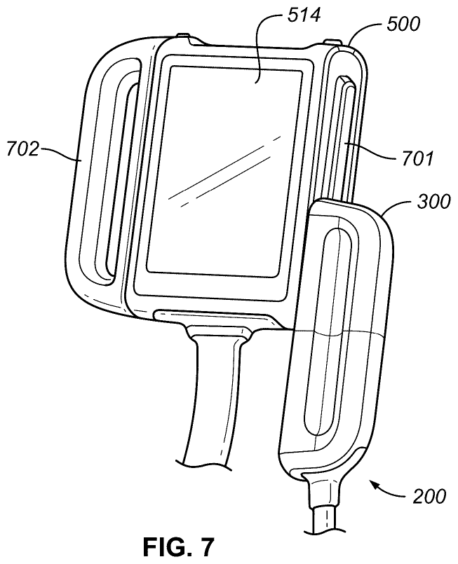

An embodiment of a system is shown in FIG. 7. The system may include an inflation cartridge 200. The inflation cartridge 200 may be configured to be removably attached to a controller 500. In some embodiments, the inflation cartridge may be slidably coupled to the controller 500. In some embodiments, the controller may further include a handle 702. In some embodiments, the controller may include a protrusion 701. In some embodiments, the external housing 300 of the inflation cartridge may include a cavity along an edge, the cavity configured to receive and latch on to the protrusion 701. In some embodiments, the system may include a display 514. The controller 500 may be configured to control the inflation cartridge. The display 514 screen may be configured to display a message. The message may instruct a user (e.g., a nurse or doctor) to load an inflation cartridge, connect pressure, prepare a catheter, insert a catheter, or connect fill lines. In some embodiments, the display 514 may show information such as the time remaining until the occlusion device is finished inflating. In other embodiments, the display 514 may show a jugular venous pressure, a right atrial pressure and the difference between the jugular venous pressure and right atrial pressure. In some embodiments, the display 514 may be configured to show the measurements in a graphical format. In some embodiments, the display 514 may instruct a user to set an occlusion volume. In some embodiments, the display 514 may be configured to receive an input from a user (i.e., a touch screen).

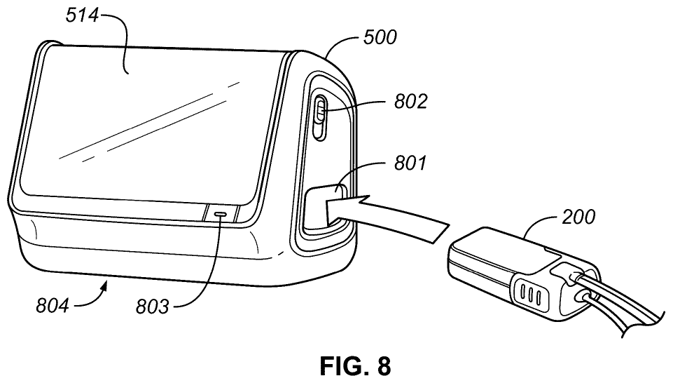

Another exemplary embodiment of a system is shown in FIG. 8. In this embodiment, the controller is shown as having a substantially quadrilateral cross-section with a substantially flat bottom surface 804. In this embodiment, the controller may be configured to slidably receive an inflation cartridge 200 through a port 801. Though FIG. 8 shows the port on the right side of the controller, one may easily envision the controller as having the port on the left side. The port 801 may include sensors, which automate the acceptance of the inflation cartridge. In this manner if the sensors detect the inflation cartridge 200 has been inserted in a correct orientation, then the sensors will automatically receive the inflation cartridge 200. However, if the inflation cartridge 200 is inserted incorrectly, then the sensors may cause the controller to reject the inflation cartridge 200. In some embodiments, the controller 500 may be configured to receive the inflation cartridge in only one orientation. In this embodiment, the controller may be coupled to a display. In this embodiment, the controller may further include a power switch 802. The power switch 802 may be configured to turn the controller on or off. The power switch 802 may further be used to turn the controller off in an emergency situation where the occlusion device must be evacuated quickly (e.g., runaway inflation of occlusion element). In this embodiment, the controller may further include an indicator light 803. The indicator light 803 may display the status of the controller 500. In some embodiments, the indicator light 803 may be green to indicate that the controller 500 is on and working properly. In some embodiments, the indicator light 803 may show another color to indicate that the device is on, but not connected to an inflation cartridge 200. In still other embodiments, the indicator light 803 may be red, to indicate that the controller 500 is either not functioning or needs service.

Still referring to FIG. 8, the controller may include a bottom surface 804. The bottom surface 804 of the controller may be flat. The flat bottom surface 804 may stand on a patient's bedside table. In some embodiment's the bottom surface 804 of the controller may be configured to stand on a flat portion of an IV stand.

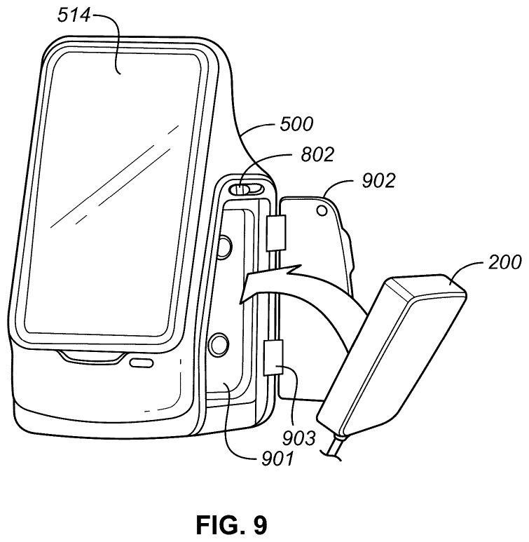

Another exemplary embodiment of the system is shown in FIG. 9. In FIG. 9, a controller 500 configured to receive an inflation cartridge 200 is shown. In some embodiments, the controller may include a side door 902. The side door 902 may be coupled to the controller via a hinge mechanism 903. The controller 500 may further include an internal housing 901. The internal housing 901 may be configured to receive an inflation cartridge 200. The internal housing 901 may be configured to receive the inflation cartridge 200 in a vertical orientation. The internal housing may be configured to include at least one opening. The at least one opening may be configured to receive at least one fluid line. Still referring to FIG. 9, the controller may further include a display 514.

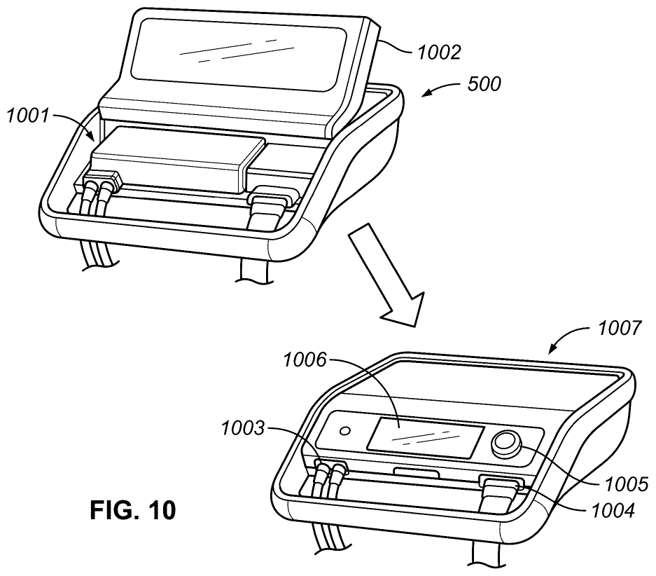

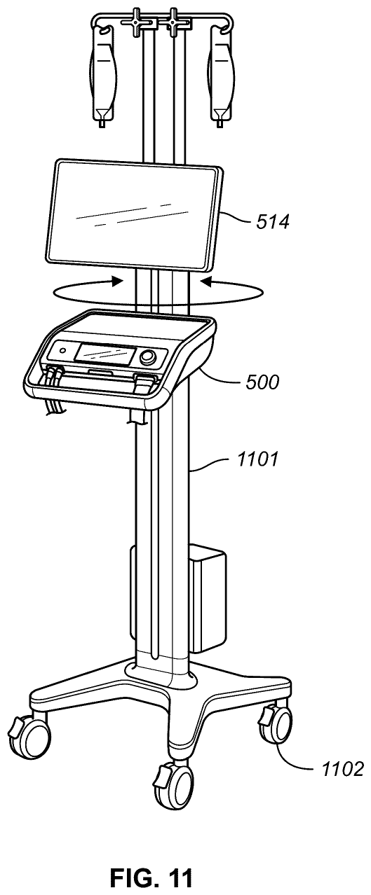

Another exemplary embodiment of the system is shown in FIG. 10. In FIG. 10, a controller is shown in both an open and closed position. The controller may include an internal cavity 1001. The internal cavity 1001 may be configured to receive an inflation cartridge 200. In this embodiment, the controller 500 may include a pivotably mounted cover 1002. The pivotably mounted cover 1002 may include at least two openings 1003, 1004. The at least two openings 1003, 1004 may be configured to receive at least two fluid lines. The at least two fluid lines may be coupled to the inflation cartridge. The controller may further include an indicator screen 1006. The indicator screen 1006 may display a message indicating whether the device is turned on or whether the fluid lines are attached properly. Although, other messages may be envisioned. The controller may further include a knob 1005. The knob 1005 may be configured to adjust the pressure of the occlusion device or the flow of the liquid. The controller may include a back portion 1007. The back portion 1007 of the controller may be operably coupled to a boom or an IV stand (as seen in FIG. 11).

Referring to FIG. 11, in some embodiments, the controller 500 may be coupled to a display 514. The display 514 may be electronically coupled to the controller 500. The controller 500 and display 514 may be pivotally mounted on a boom or IV stand 1101. The controller and display may be configured to be slidably adjusted vertically on the IV stand. In this manner, the controller 500 and display 514 may be adjusted to account for the needs of operating nurses and doctors. The IV stand may include wheels 1102. In this manner the system is portable and may travel with a patient throughout a hospital environment.

The cartridge may be configured to be slidably coupled to a console. The console may include a display, a handle, a pump, and a power source. The console may be configured to mount to an existing IV pole, a patient bedside, or on a boom.

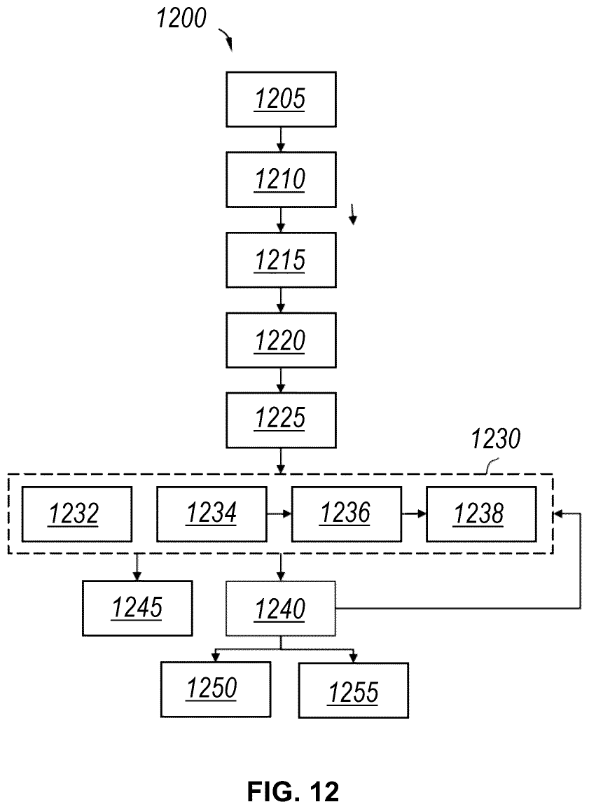

In various aspects, a method for using an expandable occlusion device may be provided. Referring to FIG. 12, the method 1200 may include removably attaching 1205 the inflation cartridge to a controller. This may include coupling the inflation cartridge to an expandable occlusion device. The inflation cartridge may be disposed outside a sterile field. The expandable occlusion device may be disposed within a sterile field.

The method may include receiving 1210 a fluid in a first syringe from a fluid source, through one or more valves of a plurality of valves on the cartridge. The fluid may be a liquid, such as a saline fluid, and may include a contrast agent.

The method may include flowing 1215 fluid from the first syringe to a second syringe through at least one of the plurality of valves. In some embodiments, the fluid may be flowed from the first syringe to the second syringe through each of the plurality of valves. In some embodiments, the fourth valve may be open to the channel coupled to the fluid sensor (see FIG. 2) until the fluid sensor detects fluid, at which time the fourth valve may be reconfigured to allow fluid to flow into the second syringe.

The method may include drawing 1220 air, with the first syringe, from an expandable occlusion device.

Drawing air from the expandable occlusion device may cause the occlusion element to enter a compressed configuration. When the occlusion element is in the compressed configuration, the occlusion device may be inserted into a patient. As such, in some embodiments, the method may include determining or confirming 1225 (e.g., via information from a pressure sensor, receiving user input, etc.) whether the expandable occlusion device was inserted into a blood vessel while the occlusion element is in the compressed configuration. This step may occur prior to flowing any fluid into the occlusion device.

The method may include flowing 1230 the fluid to the expandable occlusion device. In some embodiments, this may include causing 1232 the fluid to flow from the second syringe to the expandable occlusion device. In some embodiments, this may include purging air 1234 (e.g., flowing air from the first syringe through the first valve 230, past the fluid sensor 250, and out through valve or drain port 260). This may include flowing 1236 the fluid from the second syringe to the first syringe. This may include causing 1238 the fluid to flow from the first syringe to the expandable occlusion device.

The method may include flowing 1240 fluid from the expandable occlusion device to either the first or second syringe. The method may include repeatedly flowing fluid into and out of the expandable occlusion device at predetermined times.

The method may include intermittently providing 1245 a saline flush to the expandable occlusion device.

The method may include removing 1250 the inflation cartridge from the controller. In some embodiments, this removal may occur after a predetermined period of time. In some embodiments, this removal may occur after a predetermined number of cycles of filling and evacuating the expandable occlusion element.

The method may include determining or confirming 1255 (e.g., via information from a pressure sensor, receiving user input, etc.) whether the expandable occlusion device was removed from the blood vessel.

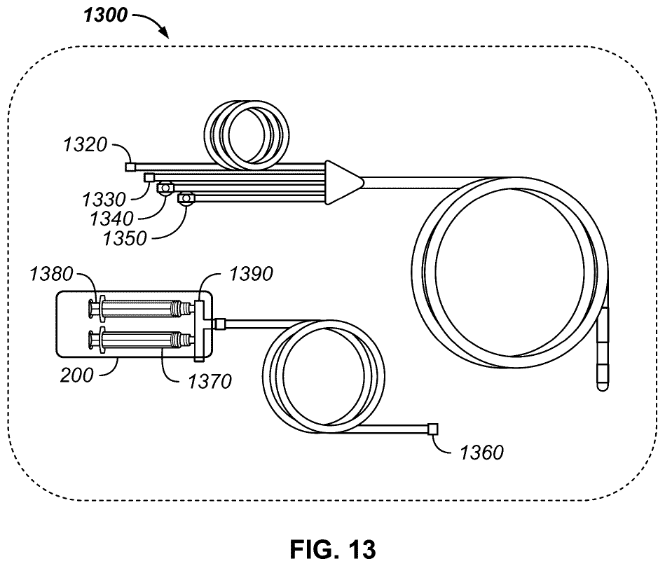

In various aspects, a kit 1300 may be provided. Referring now to FIG. 13, the kit may include an inflation cartridge 200 as disclosed herein. The kit may include one or more fill lines. The one or more fill lines may include an optical pressure lumen 1320, an occlusion balloon lumen 1330, PA/Guidewire lumen 1340, and/or floatation balloon lumen 1350. The kit may also include at least two syringes 1370, 1380. In some embodiments, the kit may also include a fluid evacuation valve 1390. In some embodiments, the kit may also include an occlusion fill line. 1360.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. An inflation cartridge, comprising:
a first syringe and a second syringe;
an external housing defining an internal volume of space, the external housing having:
at least one opening configured to allow access to an end of the first syringe and the second syringe;
a first port configured to be coupled to an expandable occlusion device; and
a second port configured to be coupled to an external fluid source; and
a plurality of valves operably coupled to the first syringe and the second syringe, the first port, and the second port.

2. The inflation cartridge of claim 1, wherein the plurality of valves comprises:
a first valve coupled to the first syringe via a first channel; and
a second valve coupled to the first valve via a second channel, the second valve also coupled to the first port.

3. The inflation cartridge of claim 2, wherein the plurality of valves further comprises a third valve coupled to the second valve via a third channel, the third valve also coupled to the second port, the third valve also coupled to a fourth valve via a fourth channel; and
wherein the fourth valve is coupled to the second syringe.

4. The inflation cartridge of claim 3, wherein the first valve and the fourth valve are also coupled to a fluid sensor via one or more additional channels.

5. The inflation cartridge of claim 1, further comprising one or more processors configured to, collectively, control the inflation cartridge.

6. The inflation cartridge of claim 1, further comprising one or more interfaces.

7-9. (canceled)

10. A system, comprising:
an inflation cartridge of claim 1; and
a controller configured to removably receive the inflation cartridge.

11. The system of claim 10, wherein the controller comprises a first actuator configured to control the first syringe, and a second actuator configured to control the second syringe.

12. The system of claim 10, wherein the external housing of the inflation cartridge is configured to be received by the controller in only one orientation.

13. The system of claim 10, further comprising an expandable occlusion device operably coupled to the inflation cartridge.

14. The system of claim 13, wherein the controller comprises one or more processors configured to, collectively, control the inflation cartridge.

15. (canceled)

16. The system of claim 14, wherein the one or more processors are configured to cause the inflation cartridge to receive fluid from a fluid source.

17. (canceled)

18. The system of claim 16, wherein the fluid received from the fluid source flows into the first syringe through one or more valves of the plurality of valves.

19. The system of claim 18, wherein the fluid is flowed from the first syringe to the second syringe through at least one of the plurality of valves.

20. The system of claim 19, wherein the fluid is flowed from the first syringe to the second syringe through each of the plurality of valves.

21. The system of claim 19, wherein the one or more processors are configured to cause the first syringe to draw air from an expandable occlusion device operably coupled to the inflation cartridge.

22. The system of claim 21, wherein the one or more processors are configured to cause the first syringe to purge air from the first syringe.

23. The system of claim 22, wherein the one or more processors are configured to cause the fluid to flow from the second syringe to the first syringe.

24-25. (canceled)

26. A method for using an expandable occlusion device, comprising:
receiving fluid in a first syringe of an inflation cartridge from a fluid source, through one or more valves of a plurality of valves;
flowing fluid from the first syringe to a second syringe through at least one of the plurality of valves;
drawing air, with the first syringe, from an expandable occlusion device; and
flowing the fluid to the expandable occlusion device.

27-40. (canceled)

41. A kit comprising:
an inflation cartridge of claim 1;
an expandable occlusion device; and
optionally one or more fill lines for coupling the inflation cartridge to the expandable occlusion device.
